(19) United States
(12) Patent Application Publication
(10) Pub. No.: US 2025/0352129 A1
(43) Pub. Date: Nov. 20, 2025

(54) PROSTATE CANCER DIAGNOSIS SYSTEM AND METHOD FOR PREDICTING PROSTATE CANCER BY DIGITALLY CONVERTING TACTILE SENSATIONS OF USER

(71) Applicant: INDUSTRIAL ACADEMIC COOPERATION FOUNDATION KEIMYUNG UNIVERSITY, Daegu (KR)

(72) Inventors: Jong-Ha LEE, Daegu (KR); So Hyun CHO, Daegu (KR)

(21) Appl. No.: 19/285,002

(22) Filed: Jul. 30, 2025

Related U.S. Application Data
(63) Continuation of application No. PCT/KR2023/018328, filed on Nov. 15, 2023.

(30) Foreign Application Priority Data
Jan. 31, 2023 (KR) 10-2023-0012416

(51) Int. Cl. A61B 5/00; A61B 5/03

(52) U.S. Cl. CPC A61B 5/4381; A61B 5/0053; A61B 5/036; A61B 2562/0247; A61B 2562/046

(57) ABSTRACT

A prostate cancer diagnosis system and method for predicting prostate cancer by digitally converting tactile sensations of a user is proposed, and according to the proposed system and method, the system is configured to include a prostate cancer measurement device configured to visualize the tactile sensation as an image by using electrodes applied according to changes in elasticity of prostate tissue to which pressure is applied, and a mobile device configured to receive visualized prostate tactile sensation numerical data from the prostate cancer measurement device, analyze presence or absence of the tissue on the basis of the prostate tactile sensation numerical data, and diagnose the prostate cancer through a result of analyzing the numerical data, so as to output the diagnosis, wherein palpation of the prostate cancer can be caused by using a small tactile sensor and easy diagnosis of an early-stage prostate nodule is possible without relying on experts.

### CROSS-REFERENCE TO RELATED APPLICATIONS

This application is a Continuation of Application No. PCT/KR2023/018328, filed on Nov. 15, 2023, which in turn claims the benefit of Korean Patent Application No. 10-2023-0012416, filed on Jan. 31, 2023. The entire disclosures of all these applications are hereby incorporated by reference.

### TECHNICAL FIELD

The present disclosure relates to a prostate cancer diagnosis system and method capable of predicting prostate cancer by digitally converting tactile sensations of a user and, more particularly, to a prostate cancer diagnosis system and method capable of predicting prostate cancer by digitally converting tactile sensations of a user, the system and method enabling an ordinary person to easily self-diagnose prostate cancer at home through palpation of the prostate cancer by using a small tactile sensor.

### BACKGROUND ART

The content described in this section simply provides background information on one exemplary embodiment of the present disclosure, and does not constitute the related art.

In the rapidly aging Korean society, one of male diseases increasing very rapidly is a prostate disease. The prostate disease such as prostate cancer, benign prostatic hyperplasia, and prostatitis usually shows mild symptoms in men in their 50s but rapidly increase in an incidence rate as the men enter their 60s and 70s, and in this regard, aging population and western eating habits are cited as the biggest causes. As methods for diagnosing the prostate diseases, there are digital rectal examinations, prostate ultrasound examinations, and the like.

A digital rectal examination is a test in which medical staff uses his or her finger inserted into the rectum of a patient to directly touch a prostate region and check the size, shape, sensation, etc. of the prostate, whereby prostate disease can be diagnosed by checking for abnormal lesions such as a hard or lumpy nodule. However, since the digital rectal examination is performed by inserting the medical staff's finger into the patient's rectum and relying on the medical staff's tactile sensation, the subjective judgement of medical staff inevitably intervenes to a significant extent. In addition, examination information cannot be quantified as well, so the reliability of the examination is somewhat low, and there are limitations in accurately transferring and sharing the examination information with other medical staff.

A prostate ultrasound examination is performed by inserting an ultrasound probe through the anus of a patient while the patient is lying on his or her side and obtaining images, and the overall shape, size, symmetry of the prostate, shape of the seminal vesicle, etc. are observed. Typical ultrasound devices capture images of the shape and size of the prostate and provide only visual information that allows determining distribution level of the blood vessels using blue and red colors. In addition, in a case of simultaneously performing a digital rectal examination and an ultrasound examination, there is a problem of causing shame and pain to a patient because an ultrasound device or medical staff's finger has to be inserted alternately into the anus of the patient.

The above-described background art is technical information that the present inventor possessed for derivation of the present disclosure or obtained in a derivation process of the present disclosure, and is not necessarily known technology disclosed to the general public prior to filing the embodiment of the present disclosure.

Korean Patent Application Publication No. 10-2021-0025398 is disclosed as the document of related art.

### DISCLOSURE

### Technical Problem

The present disclosure is proposed to solve the above-described problems of the previously proposed methods, and an objective of the present disclosure is to provide a prostate cancer diagnosis system and method capable of predicting prostate cancer by digitally converting a user's tactile sensation, the system is configured to include: a prostate cancer measurement device configured to visualize the tactile sensation as an image by using electrodes applied according to changes in elasticity of prostate tissue to which pressure is applied; and a mobile device configured to receive visualized prostate tactile sensation numerical data from the prostate cancer measurement device, analyze the presence or absence of the tissue on the basis of the prostate tactile sensation numerical data, and diagnose the prostate cancer through a result of analyzing the numerical data, so as to output the diagnosis, wherein palpation of the prostate cancer can be caused by using a small tactile sensor and easy diagnosis of an early-stage prostate nodule is possible without relying on experts.

In addition, another objective of the present disclosure is to provide a prostate cancer diagnosis system and method capable of predicting prostate cancer by digitally converting a user's tactile sensation, and in the embodiment of the present disclosure, the palpation of the prostate cancer can be caused by using a small tactile sensor, whereby the reliability of the palpation, which is the first step in a conventional prostate examination using a sensor is increased, the large cost of the prostate examination is reduced, and an early self-examination and continuous follow-up examinations may be facilitated for prostate cancer having a high incidence rate.

Furthermore, a yet another objective of the present disclosure is to provide a prostate cancer diagnosis system and method capable of predicting prostate cancer by digitally converting a user's tactile sensation, and in the embodiment of the present disclosure, the palpation and analysis of the prostate cancer is enabled by using a prostate cancer measurement device and a mobile device, so as to allow self-diagnosis of a digital rectal examination, which is used to be performed by different medical staff through a visit to a hospital, whereby voluntary and repeated screening tests may be encouraged by eliminating the screening test-associated aversion and shame felt by each patient suspected of having the prostate cancer, tumors in an early stage of development are expected to be detected through this way, and a user may easily monitor the prostate cancer anytime and anywhere by checking analysis results in real time.

However, the technical problem to be solved by the present disclosure is not limited to the above technical problems, and yet another technical problems may exist.

### Technical Solution

According to the characteristics of the present disclosure to achieve the above-described objective, there is provided a prostate cancer diagnosis system capable of predicting prostate cancer by digitally converting a user's tactile sensation, and
the prostate cancer diagnosis system includes:
a prostate cancer measurement device configured to visualize the tactile sensation as an image by using electrodes applied according to changes in elasticity of prostate tissue to which pressure is applied; and
a mobile device configured to receive visualized prostate tactile sensation numerical data from the prostate cancer measurement device, analyze presence or absence of the tissue on the basis of the prostate tactile sensation numerical data, and diagnose the prostate cancer through a result of analyzing the numerical data, so as to output the diagnosis.

Preferably, the prostate cancer measurement device may be configured to include:
a tactile sensor for detecting the tactile sensation according to the changes in the elasticity of the prostate tissue to which the pressure is applied for self-diagnosis of the prostate cancer;
a camera for photographing a region where the tactile sensation is detected by the tactile sensor according to the changes in the elasticity of the prostate tissue to which the pressure is applied, so as to obtain a tactile sensation image;
a Bluetooth module for transmitting the prostate tactile sensation numerical data obtained through the tactile sensor and the camera to the mobile device; and
a pressure numerical determination algorithm for determining a pressure numerical value of the tactile sensation according to the changes in the elasticity of the prostate tissue detected by the tactile sensor.

More preferably, the tactile sensor
may detect the tactile sensation according to the changes in the elasticity of the prostate tissue to which the pressure is applied for the self-diagnosis of the prostate cancer, and due to contacting a lump during normal probing in which uniform electrical signals are detected, detect a region where the uniform electrical signals are changed into abnormal signals as a prostate nodule region.

Even more preferably, the tactile sensor
may configured in an elastic optical waveguide structure in which four white LED units are attached to a glass plate to illuminate an optical waveguide.

Even more preferably, the camera
may be configured as a near-infrared camera (NIR Camera) that obtains the tactile sensation image by photographing the region where the tactile sensation is detected by the tactile sensor according to the changes in the elasticity of the prostate tissue to which the pressure is applied.

Even more preferably, the Bluetooth module
may transmit the prostate tactile sensation numerical data obtained through the tactile sensor and camera to the mobile device in real time.

Preferably, the mobile device may be configured to include:
a receiving module for receiving the visualized prostate tactile sensation numerical data from the prostate cancer measurement device;
a determination analysis module for analyzing the presence or absence of the tissue on the basis of the prostate tactile sensation numerical data received through the receiving module; and
an output module for diagnosing the prostate cancer on the basis of the result of analyzing the numerical data analyzed through the determination analysis module, so as to output the diagnosis.

More preferably, the mobile device
may output a notification indicating a risk through the output module when the diagnosis of the prostate cancer is determined through the result of analyzing the numerical data analyzed by the determination analysis module.

According to the characteristics of the present disclosure to achieve the above-described objective, there is provided a prostate cancer diagnosis method capable of predicting prostate cancer by digitally converting a user's tactile sensation,
the prostate cancer diagnosis method includes:
(1) visualizing, by a prostate cancer measurement device, the tactile sensation as an image by using electrodes applied according to changes in elasticity of prostate tissue to which pressure is applied; and
(2) receiving, by a mobile device, visualized prostate tactile sensation numerical data from the prostate cancer measurement device, analyzing presence or absence of the tissue on the basis of the prostate tactile sensation numerical data, and diagnosing the prostate cancer through a result of analyzing the numerical data, so as to output the diagnosis.

Preferably, the prostate cancer measurement device may be configured to include:
a tactile sensor for detecting the tactile sensation according to the changes in the elasticity of the prostate tissue to which the pressure is applied for self-diagnosis of the prostate cancer;
a camera for photographing a region where the tactile sensation is detected by the tactile sensor according to the changes in the elasticity of the prostate tissue to which the pressure is applied, so as to obtain a tactile sensation image;
a Bluetooth module for transmitting the prostate tactile sensation numerical data obtained through the tactile sensor and the camera to the mobile device; and
a pressure numerical determination algorithm for determining a pressure numerical value of the tactile sensation according to the changes in the elasticity of the prostate tissue detected by the tactile sensor.

More preferably, the tactile sensor
may detect the tactile sensation according to the changes in the elasticity of the prostate tissue to which the pressure is applied for the self-diagnosis of the prostate cancer, and due to contacting a lump during normal probing in which uniform electrical signals are detected, detect a region where the uniform electrical signals are changed into abnormal signals as a prostate nodule region.

Even more preferably, the tactile sensor
may be configured in an elastic optical waveguide structure in which four white LED units are attached to a glass plate to illuminate an optical waveguide.

Even more preferably, the camera
may be configured as a near-infrared camera (NIR Camera) that obtains the tactile sensation image by photographing the region where the tactile sensation is detected by the tactile sensor according to the changes in the elasticity of the prostate tissue to which the pressure is applied.

Even more preferably, the Bluetooth module
may transmit the prostate tactile sensation numerical data obtained through the tactile sensor and camera to the mobile device in real time.

Preferably, the mobile device may be configured to include:
a receiving module for receiving the visualized prostate tactile sensation numerical data from the prostate cancer measurement device;
a determination analysis module for analyzing the presence or absence of the tissue on the basis of the prostate tactile sensation numerical data received through the receiving module; and
an output module for diagnosing the prostate cancer on the basis of the result of analyzing the numerical data analyzed through the determination analysis module, so as to output the diagnosis.

More preferably, the mobile device
may output a notification indicating a risk through the output module when the diagnosis of the prostate cancer is determined through the result of analyzing the numerical data analyzed by the determination analysis module.

### Advantageous Effects

According to the present disclosure proposing the prostate cancer diagnosis system and method capable of predicting the prostate cancer by digitally converting the user's tactile sensation, the system is configured to include: a prostate cancer measurement device configured to visualize the tactile sensation as an image by using electrodes applied according to changes in elasticity of prostate tissue to which pressure is applied; and a mobile device configured to receive visualized prostate tactile sensation numerical data from the prostate cancer measurement device, analyze the presence or absence of the tissue on the basis of the prostate tactile sensation numerical data, and diagnose the prostate cancer through a result of analyzing the numerical data, so as to output the diagnosis, wherein palpation of the prostate cancer can be caused by using a small tactile sensor and easy diagnosis of an early-stage prostate nodule is possible without relying on experts.

In addition, according to the present disclosure proposing the prostate cancer diagnosis system and method capable of predicting the prostate cancer by digitally converting the user's tactile sensation, the palpation of the prostate cancer can be caused by using a small tactile sensor, whereby the reliability of the palpation, which is the first step in a conventional prostate examination using a sensor is increased, the large cost of the prostate examination is reduced, and an early self-examination and continuous follow-up examinations may be facilitated for the prostate cancer having a high incidence rate.

Furthermore, according to the present disclosure proposing the prostate cancer diagnosis system and method capable of predicting the prostate cancer by digitally converting the user's tactile sensation, the palpation and analysis of the prostate cancer is enabled by using a prostate cancer measurement device and a mobile device, so as to allow self-diagnosis of a digital rectal examination, which is used to be performed by different medical staff through a visit to a hospital, whereby voluntary and repeated screening tests may be encouraged by eliminating the screening test-associated aversion and shame felt by each patient suspected of having the prostate cancer, tumors in an early stage of development are expected to be detected through this way, and a user may easily monitor the prostate cancer anytime and anywhere by checking analysis results in real time.

In addition, the various and advantageous strong points and effects of the present disclosure are not limited to the above-described content, and will be more easily understood in the process of describing the specific embodiment of the present disclosure.

### DESCRIPTION OF DRAWINGS

FIG. 1 is a view illustrating a configuration, shown in functional blocks, of a prostate cancer diagnosis system capable of predicting prostate cancer by digitally converting a user's tactile sensation according to one exemplary embodiment of the present disclosure.

FIG. 2 is a view illustrating a configuration, shown in functional blocks, of a prostate cancer measurement device of the prostate cancer diagnosis system capable of predicting the prostate cancer by digitally converting the user's tactile sensation according to one exemplary embodiment of the present disclosure.

FIG. 3 is a view illustrating a configuration, shown in functional blocks, of a mobile device of the prostate cancer diagnosis system capable of predicting the prostate cancer by digitally converting the user's tactile sensation according to one exemplary embodiment of the present disclosure.

FIG. 4 is a view illustrating the schematic flow of a prostate cancer numerical determination algorithm of the prostate cancer diagnosis system capable of predicting the prostate cancer by digitally converting the user's tactile sensation according to one exemplary embodiment of the present disclosure.

FIG. 5 is a view illustrating a configuration of an example of a tactile sensor of the prostate cancer diagnosis system capable of predicting the prostate cancer by digitally converting the user's tactile sensation according to one exemplary embodiment of the present disclosure.

FIG. 6 is a view illustrating processing of an imaging process of the prostate cancer diagnosis system capable of predicting the prostate cancer by digitally converting the user's tactile sensation according to one exemplary embodiment of the present disclosure.

FIG. 7 is a view illustrating pictures each obtained by extracting a final prostate determination region by an algorithm that extracts a nodule region from a prostate image of the prostate cancer diagnosis system capable of predicting the prostate cancer by digitally converting the user's tactile sensation according to one exemplary embodiment of the present disclosure.

FIG. 8 is a view illustrating the flow of a prostate cancer diagnosis method capable of predicting prostate cancer by digitally converting a user's tactile sensation according to one exemplary embodiment of the present disclosure.

FIG. 9 is a view illustrating the detailed flow of the prostate cancer diagnosis method capable of predicting the prostate cancer by digitally converting the user's tactile sensation according to one exemplary embodiment of the present disclosure.

### DESCRIPTION OF THE REFERENCE NUMERALS IN THE DRAWINGS

100: prostate cancer diagnosis system according to one exemplary embodiment of the present disclosure
110: prostate cancer measurement device
111: tactile sensor
112: camera
113: Bluetooth module
114: pressure numerical determination algorithm
120: mobile device
121: receiving module
122: determination analysis module
123: output module
S110: visualizing, by prostate cancer measurement device using tactile sensor, tactile sensation as image by using electrodes applied according to changes in elasticity of prostate tissue to which pressure is applied
S120: receiving, by mobile device, visualized prostate tactile sensation numerical data from prostate cancer measurement device, analyzing presence or absence of tissue on basis of prostate tactile sensation numerical data, and diagnosing prostate cancer through result of analyzing numerical data, so as to output diagnosis
S121: receiving prostate visualized tactile sensation numerical data
S122: analyzing presence or absence of tissue on basis of prostate tactile sensation numerical data
S123: outputting result of analyzing numerical data

### BEST MODE

Hereinafter, one exemplary embodiment of the present disclosure will be described in detail with reference to the accompanying drawings so that those skilled in the art may easily implement the present disclosure. However, the present disclosure is not limited to the exemplary embodiment described herein and may be embodied in many different forms. In addition, in order to clearly describe the present disclosure in the drawings, parts irrelevant to the description are omitted, and like reference numerals designate like elements throughout the specification.

Throughout the present specification, when a part is said to be “connected” to another part, an expression such as “connected” is intended to include not only “directly connected” but also “indirectly connected”, while having a different element in the middle thereof. In addition, it will be further understood that, when a part is said to “include” or “comprise” a certain component, it means that it may further include or comprise other components, but does not exclude other components unless the context clearly indicates otherwise, and does not preclude the presence or addition of one or more other features, integers, steps, operations, elements, components, and/or combinations thereof.

The following exemplary embodiment is a detailed description for better understanding of the embodiment of the present disclosure, and do not limit the scope of the present disclosure. Therefore, embodiments of the same scope that perform the same functions as those of the present disclosure will also fall within the scope of the present disclosure.

In addition, each component, process, progress, method, or the like included in each exemplary embodiment of the present disclosure may be shared within a range that does not contradict each other technically.

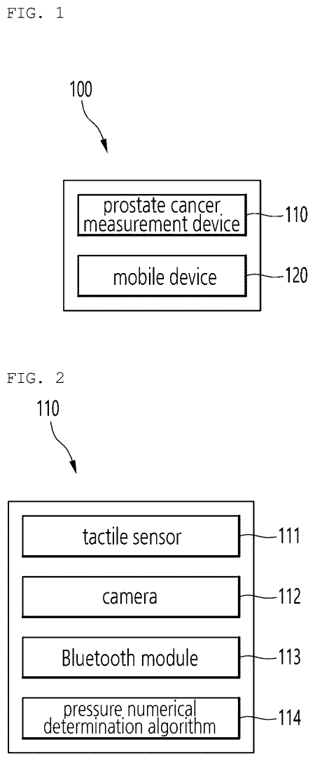

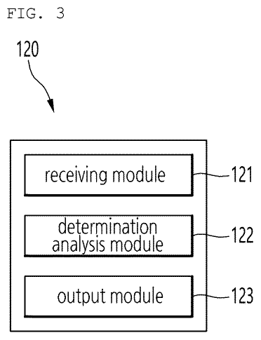

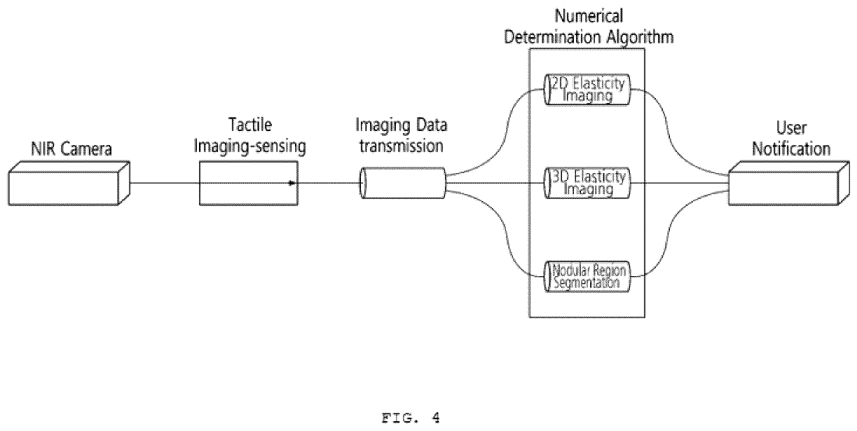

FIG. 1 is a view illustrating a configuration, shown in functional blocks, of a prostate cancer diagnosis system capable of predicting prostate cancer by digitally converting a user's tactile sensation according to one exemplary embodiment of the present disclosure. FIG. 2 is a view illustrating a configuration, shown in functional blocks, of a prostate cancer measurement device of the prostate cancer diagnosis system capable of predicting the prostate cancer by digitally converting the user's tactile sensation according to one exemplary embodiment of the present disclosure. FIG. 3 is a view illustrating a configuration, shown in functional blocks, of a mobile device of the prostate cancer diagnosis system capable of predicting the prostate cancer by digitally converting the user's tactile sensation according to one exemplary embodiment of the present disclosure. As illustrated in each of FIGS. 1 to 3, the system may be configured to include: a prostate cancer measurement device 110 configured to visualize the tactile sensation as an image by using electrodes applied according to changes in elasticity of prostate tissue to which pressure is applied; and a mobile device 120 configured to receive visualized prostate tactile sensation numerical data from the prostate cancer measurement device 110, analyze the presence or absence of the tissue on the basis of the prostate tactile sensation numerical data, and diagnose the prostate cancer through a result of analyzing the numerical data, so as to output the diagnosis. Hereinafter, with reference to the attached drawings, a detailed description will be given of a specific configuration of the prostate cancer diagnosis system capable of predicting the prostate cancer by digitally converting the user's tactile sensation according to one exemplary embodiment of the present disclosure.

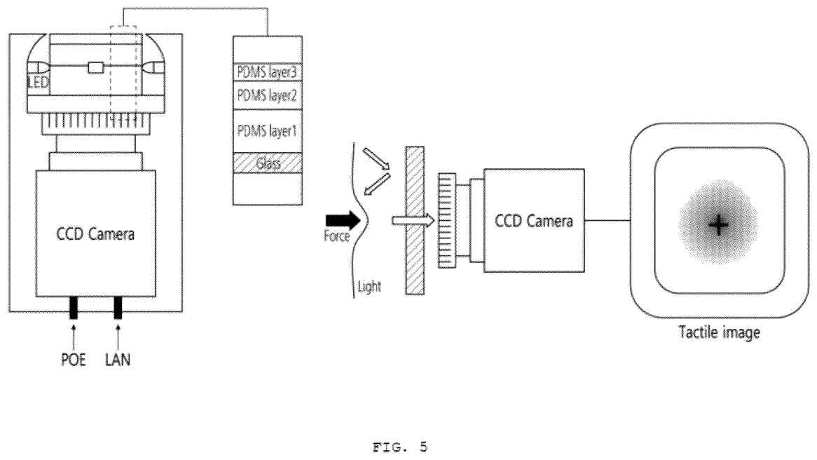

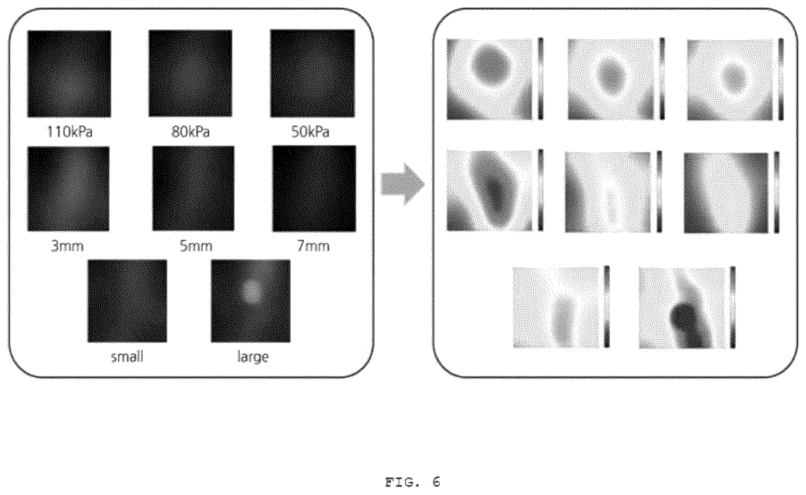

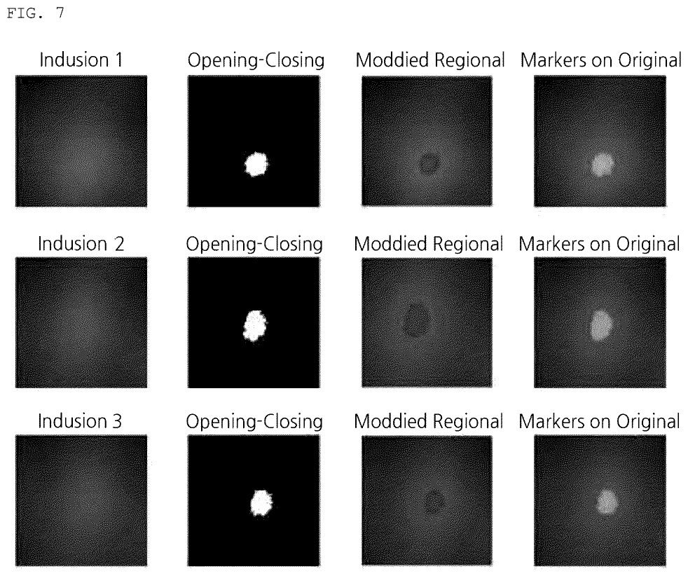

FIG. 4 is a view illustrating the schematic flow of a prostate cancer numerical determination algorithm of the prostate cancer diagnosis system capable of predicting the prostate cancer by digitally converting the user's tactile sensation according to one exemplary embodiment of the present disclosure. FIG. 5 is a view illustrating a configuration of an example of a tactile sensor of the prostate cancer diagnosis system capable of predicting the prostate cancer by digitally converting the user's tactile sensation according to one exemplary embodiment of the present disclosure. FIG. 6 is a view illustrating processing of an imaging process of the prostate cancer diagnosis system capable of predicting the prostate cancer by digitally converting the user's tactile sensation according to one exemplary embodiment of the present disclosure. FIG. 7 is a view illustrating pictures each obtained by extracting a final prostate determination region by an algorithm that extracts a nodule region from a prostate image of the prostate cancer diagnosis system capable of predicting the prostate cancer by digitally converting the user's tactile sensation according to one exemplary embodiment of the present disclosure.

The prostate cancer measurement device 110 is a component for visualizing a tactile sensation as an image by using electrodes applied according to changes in elasticity of prostate tissue to which pressure is applied. As illustrated in FIG. 2, such a prostate cancer measurement device 110 may be configured to include: a tactile sensor 111 for detecting a tactile sensation according to the changes in the elasticity of the prostate tissue to which the pressure is applied for self-diagnosis of prostate cancer; a camera 112 for photographing a region where the tactile sensation is detected by the tactile sensor 111 according to the changes in the elasticity of the prostate tissue to which the pressure is applied, so as to obtain a tactile sensation image; a Bluetooth module 113 for transmitting prostate tactile sensation numerical data obtained through the tactile sensor 111 and the camera 112 to the mobile device 120; and a pressure numerical determination algorithm 114 for determining a pressure numerical value of the tactile sensation according to the changes in the elasticity of the prostate tissue detected by the tactile sensor 111.

In addition, the tactile sensor 111 may detect the tactile sensation according to the changes in the elasticity of the prostate tissue to which the pressure is applied for the self-diagnosis of the prostate cancer, and due to contacting a lump during normal probing in which uniform electrical signals are detected, detects a region where the uniform electrical signals are changed into abnormal signals as a prostate nodule region. Such a tactile sensor 111 may be configured in an elastic optical waveguide structure in which four white LED units are attached to a glass plate to illuminate an optical waveguide. Here, the tactile sensor 111 is a small sensor that may utilize the principle that when a patient's skin is probed and a lump is contacted, the returning electrical signals are no longer uniform and come in with a large amount more than a certain value. That is, as illustrated in FIG. 5, the tactile sensor 111 may be configured to have a three-layer structure similar to a patient's skin by laminating silicone layers having strengths corresponding to the dermis, epidermis, and subcutaneous fat, respectively, in an elastic optical waveguide unit structure.

In addition, the camera 112 may be configured as a near-infrared camera (NIR Camera) for obtaining a tactile sensation image by photographing a region where a tactile sensation is detected by the tactile sensor 111 according to changes in elasticity of prostate tissue to which pressure is applied. Such a camera 112 may capture the tactile sensation image of a tactile sensation detected according to the changes in the elasticity of the prostate tissue to which the pressure is applied by the tactile sensor 111, i.e., a tactile sensation of an abnormal region suspected of being a prostate nodule. That is, the waveguide is soft and elastic, so when the waveguide is compressed by an external force from a solid object, a contact region of the waveguide is deformed, causing light to be scattered, and the scattered light is captured by a high-resolution camera 112 and stored in image format. The fundamental principle of tactile imaging may be implemented in a method of monitoring the scattering of light that occurs when a critical angle is changed by the external force.

In addition, the Bluetooth module 113 may transmit prostate tactile sensation numerical data obtained through the tactile sensor 111 and camera 112 to the mobile device 120 in real time. Such a Bluetooth module 113 performs a communication function for transmission and reception with the receiving module 121 of the mobile device 120. The prostate cancer measurement device 110 may also be interconnected in a wired manner through a cable connection, in addition to Bluetooth communication through the Bluetooth module 113.

In addition, the pressure numerical determination algorithm 114 may be used for determining a pressure numerical value of tactile sensation according to changes in elasticity of prostate tissue detected by the tactile sensor 111 and transmit it to the mobile device 120 through the Bluetooth module 113, or may be operated as a control function for obtaining a tactile sensation image through the camera 112. Such a pressure numerical determination algorithm 114 may determine the presence or absence of an abnormality in the tactile sensation detected through the tactile sensor 111 and may function to control data transmission to the mobile device 120 therethrough. That is, the pressure numerical determination algorithm 114 may function to determine a pressure numerical value of the detected tactile sensation of the tactile sensor 111 and to be interconnected with the mobile device 120.

The mobile device 120 is configured to receive visualized prostate tactile sensation numerical data from the prostate cancer measurement device 110, analyze the presence or absence of tissue on the basis of the prostate tactile sensation numerical data, and diagnose prostate cancer on the basis of a result of analyzing the numerical data, so as to output the diagnosis. As illustrated in FIG. 3, such a mobile device 120 may be configured to include: a receiving module 121 for receiving the visualized prostate tactile sensation numerical data from the prostate cancer measurement device 110; a determination analysis module 122 for analyzing the presence or absence of the tissue on the basis of the prostate tactile sensation numerical data received through the receiving module 121; and an output module 123 for diagnosing the prostate cancer on the basis of the result of analyzing the numerical data analyzed through the determination analysis module 122, so as to output the diagnosis.

In addition, the mobile device 120 may output a notification indicating a risk through the output module 123 when the diagnosis of the prostate cancer is determined through the result of analyzing the numerical data analyzed by the determination analysis module.

In addition, the determination analysis module 122 is provided with an algorithm for extracting a nodule region from a prostate image, and may function to extract a final prostate nodule region so as to perform a diagnosis accurately even in locations and sizes where diagnosis is uncertain. Such a determination analysis module 122 may function to immediately reduce diagnosis time and cost by extracting the nodule region from the prostate image through the prostate cancer extraction algorithm. Here, a fuzzy binary technique is used for brightness adjustment using images captured from three phantoms, a contour tracing technique is used for extracting a prostate nodule candidate region, and an erosion and dilation technique is used for extracting the final prostate nodule region.

In addition, since the image captured by the prostate cancer measurement device 110 has a bell-shaped form from which light spreads out, excessive segmentation occurs when processed without an existing preprocessing process, making it difficult to extract an exact region. Accordingly, the measured image is supplemented by reconstructing it according to a morphology technique, an empty space in the region suspected of being a nodule is filled, adjacent regions are connected to organize an outline, and objects in contact with the outline are removed in order to extract the nodule region more clearly. Using such a method, an unrequired region inside the object is removed so as to make a candidate nodule region clearly visible, and the nodule region is handled to be accurately extracted even in locations and sizes where skin texture is unable to be diagnosed.

In addition, the mobile device 120 may be any type of mobile smart device such as a smartphone, a smart note, a tablet PC, a smart camera, and a wearable computer. However, the mobile device 120 of the present disclosure is not limited to the form of the terminals as listed above, and as long as it is able to receive and analyze a prostate tactile sensation image in conjunction with the prostate cancer measurement device 110 according to one exemplary embodiment of the present disclosure, any terminal may serve as the mobile device 120 of the present disclosure regardless of a specific form of the terminal.

In addition, the mobile device 120 may operate an application for implementing the prostate cancer diagnosis method capable of predicting the prostate cancer by digitally converting the user's tactile sensation according to one exemplary embodiment of the present disclosure. At this time, the application running on the mobile device 120 may be an executable program installed by an installation program managed by an application server operating on a communication network, etc., and may also be a program executed through a network such as the Internet. The application running on such a mobile device 120 may provide various interfaces capable of monitoring prostate cancer easily.

FIG. 4 illustrates a schematic flow of a prostate cancer numerical determination algorithm of the prostate cancer diagnosis system capable of predicting the prostate cancer by digitally converting the user's tactile sensation according to one exemplary embodiment of the present disclosure. That is, FIG. 4 illustrates the flow related to a near-infrared camera, tactile sensation image detection, image data transmission, a numerical determination algorithm, and a user notification.

FIG. 5 illustrates a configuration, shown in functional blocks, of an example of a tactile sensor of the prostate cancer diagnosis system capable of predicting the prostate cancer by digitally converting the user's tactile sensation according to one exemplary embodiment of the present disclosure. That is, FIG. 5 illustrates a structure of the tactile sensor in a form in which four white LED light source units are attached to a glass plate as elastic optical waveguide units to illuminate an optical waveguide, and a camera 112 and a pressure numerical determination algorithm 114 may be configured in connection.

FIG. 6 illustrates processing of an imaging process of the prostate cancer diagnosis system capable of predicting the prostate cancer by digitally converting the user's tactile sensation according to one exemplary embodiment of the present disclosure. That is, FIG. 6 illustrates the processing of the imaging process of optical tactile sensation images, in which prostate tissue is detected, tactile data is obtained by using two-dimensional (2D) color simulation, the tactile data is visualized as an image by using three-dimensional (3D) modeling, and a tumor is explored with a 3D elasticity algorithm to enable more intuitive representation.

FIG. 7 illustrates pictures each obtained by extracting a final prostate determination region by an algorithm that extracts a nodule region from each prostate image of the prostate cancer diagnosis system capable of predicting the prostate cancer by digitally converting the user's tactile sensation according to one exemplary embodiment of the present disclosure. That is, FIG. 7 illustrates the algorithm for extracting the nodule region from each prostate image, enabling diagnosis to be performed accurately by extracting a final prostate nodule region even in locations and sizes where diagnosis is uncertain.

FIG. 8 is a view illustrating the flow of a prostate cancer diagnosis method capable of predicting prostate cancer by digitally converting a user's tactile sensation according to one exemplary embodiment of the present disclosure. As illustrated in FIG. 8, the prostate cancer diagnosis method capable of predicting the prostate cancer by digitally converting the user's tactile sensation may be implemented to include: step S110 of visualizing, by a prostate cancer measurement device 110, the tactile sensation as an image by using electrodes applied according to changes in elasticity of prostate tissue to which pressure is applied; and step S120 of receiving, by a mobile device 120, visualized prostate tactile sensation numerical data from the prostate cancer measurement device 120, analyzing the presence or absence of the tissue on the basis of the prostate tactile sensation numerical data, and diagnosing prostate cancer through a result of analyzing the numerical data, so as to output the diagnosis.

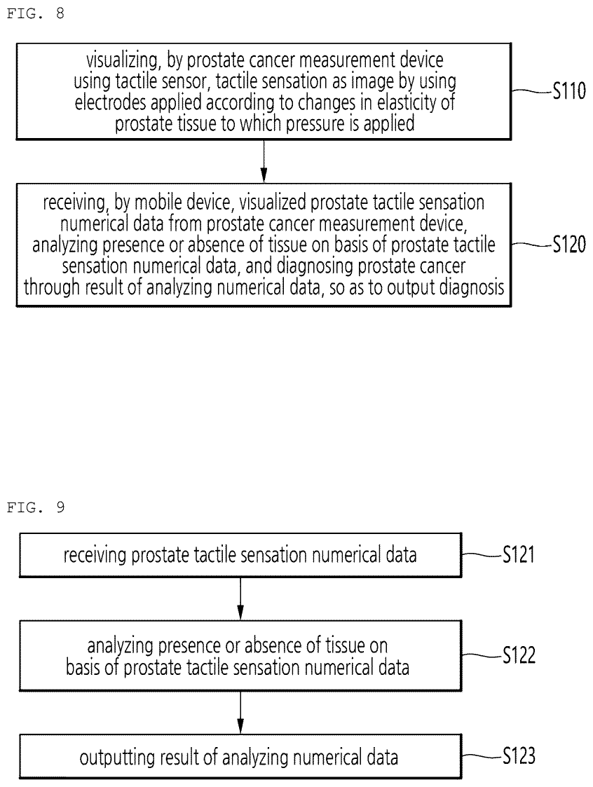

FIG. 9 is a view illustrating the detailed flow of the prostate cancer diagnosis method capable of predicting the prostate cancer by digitally converting the user's tactile sensation according to one exemplary embodiment of the present disclosure. As shown FIG. 9, this view illustrates the detailed flow of the prostate cancer diagnosis method capable of predicting the prostate cancer by digitally converting the user's tactile sensation, and this method may be implemented to include: step S121 of receiving, by the mobile device 120, the visualized prostate tactile sensation numerical data from the prostate cancer measurement device 110; step S122 of analyzing the presence or absence of the tissue on the basis of the prostate tactile sensation numerical data; and step S123 of outputting result of analyzing numerical data.

In step S110, the prostate cancer measurement device 110 visualizes the tactile sensation as the image by using the electrodes applied according to the changes in the elasticity of the prostate tissue to which the pressure is applied. As illustrated in FIG. 2, the prostate cancer measurement device 110 in such step S110 may be configured to include: a tactile sensor 111 for detecting the tactile sensation according to the changes in the elasticity of the prostate tissue to which the pressure is applied for the self-diagnosis of prostate cancer; a camera 112 for photographing a region where the tactile sensation is detected by the tactile sensor 111 according to the changes in the elasticity of the prostate tissue to which the pressure is applied, so as to obtain the tactile sensation image; a Bluetooth module 113 for transmitting prostate tactile sensation numerical data obtained through the tactile sensor 111 and the camera 112 to a mobile device 120; and a pressure numerical determination algorithm 114 for determining a pressure numerical value of the tactile sensation according to the changes in the elasticity of the prostate tissue detected by the tactile sensor 111.

In addition, the tactile sensor 111 may detect the tactile sensation according to the changes in the elasticity of the prostate tissue to which the pressure is applied for the self-diagnosis of the prostate cancer, and due to contacting a lump during normal probing in which uniform electrical signals are detected, detects a region where the uniform electrical signals are changed into abnormal signals as a prostate nodule region. Such a tactile sensor 111 may be configured in an elastic optical waveguide structure in which four white LED units are attached to a glass plate to illuminate an optical waveguide. Here, the tactile sensor 111 is a small sensor that may utilize the principle that when a patient's skin is probed and a lump is contacted, the returning electrical signals are no longer uniform and come in with a large amount more than a certain value. That is, as illustrated in FIG. 5, the tactile sensor 111 may be configured to have a three-layer structure similar to a patient's skin by laminating silicone layers having strengths corresponding to the dermis, epidermis, and subcutaneous fat, respectively, in an elastic optical waveguide unit structure.

In addition, the camera 112 may be configured as a near-infrared camera (NIR Camera) for obtaining a tactile sensation image by photographing a region where a tactile sensation is detected by the tactile sensor 111 according to changes in elasticity of prostate tissue to which pressure is applied. Such a camera 112 may capture the tactile sensation image of a tactile sensation detected according to the changes in the elasticity of the prostate tissue to which the pressure is applied by the tactile sensor 111, i.e., a tactile sensation of an abnormal region suspected of being a prostate nodule. That is, the waveguide is soft and elastic, so when the waveguide is compressed by an external force from a solid object, a contact region of the waveguide is deformed, causing light to be scattered, and the scattered light is captured by a high-resolution camera 112 and stored in image format. The fundamental principle of tactile imaging may be implemented in a method of monitoring the scattering of light that occurs when a critical angle is changed by the external force.

In addition, the Bluetooth module 113 may transmit prostate tactile sensation numerical data obtained through the tactile sensor 111 and camera 112 to the mobile device 120 in real time. Such a Bluetooth module 113 performs a communication function for transmission and reception with the receiving module 121 of the mobile device 120. The prostate cancer measurement device 110 may also be interconnected in a wired manner through a cable connection, in addition to Bluetooth communication through the Bluetooth module 113.

In addition, the pressure numerical determination algorithm 114 may be used for determining a pressure numerical value of tactile sensation according to changes in elasticity of prostate tissue detected by the tactile sensor 111 and transmit it to the mobile device 120 through the Bluetooth module 113, or may be operated as a control function for obtaining a tactile sensation image through the camera 112. Such a pressure numerical determination algorithm 114 may determine the presence or absence of an abnormality in the tactile sensation detected through the tactile sensor 111 and may function to control data transmission to the mobile device 120 therethrough. That is, the pressure numerical determination algorithm 114 may function to determine a pressure numerical value of the detected tactile sensation of the tactile sensor 111 and to be interconnected with the mobile device 120.

In step S120, the mobile device 120 is configured to receive visualized prostate tactile sensation numerical data from the prostate cancer measurement device 110, analyze the presence or absence of tissue on the basis of the prostate tactile sensation numerical data, and diagnose prostate cancer on the basis of a result of analyzing the numerical data, so as to output the diagnosis. As illustrated in FIG. 3, the mobile device 120 in such step S120 may be configured to include: a receiving module 121 for receiving the visualized prostate tactile sensation numerical data from the prostate cancer measurement device 110; a determination analysis module 122 for analyzing the presence or absence of the tissue on the basis of the prostate tactile sensation numerical data received through the receiving module 121; and an output module 123 for diagnosing the prostate cancer on the basis of a result of analyzing the numerical data analyzed through the determination analysis module 122, so as to output the diagnosis.

In addition, the mobile device 120 may output a notification indicating a risk through the output module 123 when the diagnosis of the prostate cancer is determined through the result of analyzing the numerical data analyzed by the determination analysis module.

In addition, the determination analysis module 122 is provided with an algorithm for extracting a nodule region from a prostate image, and may function to extract a final prostate nodule region so as to perform a diagnosis accurately even in locations and sizes where diagnosis is uncertain. Such a determination analysis module 122 may function to immediately reduce diagnosis time and cost by extracting the nodule region from the prostate image through the prostate cancer extraction algorithm. Here, a fuzzy binary technique is used for brightness adjustment using images captured from three phantoms, a contour tracing technique is used for extracting a prostate nodule candidate region, and an erosion and dilation technique is used for extracting the final prostate nodule region.

In addition, since the image captured by the prostate cancer measurement device 110 has a bell-shaped form from which light spreads out, excessive segmentation occurs when processed without an existing preprocessing process, making it difficult to extract an exact region. Accordingly, the measured image is supplemented by reconstructing it according to a morphology technique, an empty space in the region suspected of being a nodule is filled, adjacent regions are connected to organize an outline, and objects in contact with the outline are removed in order to extract the nodule region more clearly. Using such a method, an unrequired region inside the object is removed so as to make a candidate nodule region clearly visible, and the nodule region is handled to be accurately extracted even in locations and sizes where skin texture is unable to be diagnosed.

In addition, the mobile device 120 may be any type of mobile smart device such as a smartphone, a smart note, a tablet PC, a smart camera, and a wearable computer. However, the mobile device 120 of the present disclosure is not limited to the form of the terminals as listed above, and as long as it is able to receive and analyze a prostate tactile sensation image in conjunction with the prostate cancer measurement device 110 according to one exemplary embodiment of the present disclosure, any terminal may serve as the mobile device 120 of the present disclosure regardless of a specific form of the terminal.

In addition, the mobile device 120 may operate an application for implementing the prostate cancer diagnosis method capable of predicting the prostate cancer by digitally converting the user's tactile sensation according to one exemplary embodiment of the present disclosure. At this time, the application running on the mobile device 120 may be an executable program installed by an installation program managed by an application server operating on a communication network, etc., and may also be a program executed through a network such as the Internet. The application running on such a mobile device 120 may provide various interfaces capable of monitoring prostate cancer easily.

Meanwhile, the present disclosure may include a computer-readable medium including program instructions for performing operations implemented in various communication terminals. For example, the computer-readable media may include hardware devices specially configured to store and execute program instructions, the hardware devices including: magnetic media such as hard disks, floppy disks, and magnetic tapes; optical media such as CD_ROMs and DVDs; magneto-optical media such as floptical disks; and memories such as ROMs, RAMs, and flash memories.

The computer-readable media may include program instructions, data files, data structures, etc. individually or in combination thereof. In this case, the program instructions recorded on the computer-readable media may be specially designed and configured to implement the embodiment of the present disclosure, or may be known and available to those skilled in the art of computer software. For example, the computer instructions may include not only machine language code such as one generated by a compiler, but also high-level language code executable by a computer using an interpreter or the like.

According to the exemplary embodiments of the present disclosure as described above, there is provided the prostate cancer diagnosis system and method capable of predicting the prostate cancer by digitally converting the user's tactile sensation, the system is configured to include: a prostate cancer measurement device configured to visualize the tactile sensation as an image by using electrodes applied according to changes in elasticity of prostate tissue to which pressure is applied; and a mobile device configured to receive visualized prostate tactile sensation numerical data from the prostate cancer measurement device, analyze the presence or absence of the tissue on the basis of the prostate tactile sensation numerical data, and diagnose the prostate cancer through a result of analyzing the numerical data, so as to output the diagnosis, whereby palpation of the prostate cancer can be caused by using a small tactile sensor and easy diagnosis of an early-stage prostate nodule is possible without relying on experts. In addition, the palpation of the prostate cancer can be caused by using a small tactile sensor, whereby the reliability of the palpation, which is the first step in a conventional prostate examination using a sensor is increased, the large cost of the prostate examination is reduced, and an early self-examination and continuous follow-up examinations may be facilitated for the prostate cancer having a high incidence rate. In particular, the palpation and analysis of the prostate cancer is enabled by using a prostate cancer measurement device and a mobile device, so as to allow self-diagnosis of a digital rectal examination, which is used to be performed by different medical staff through a visit to a hospital, wherein voluntary and repeated screening tests may be encouraged by eliminating the screening test-associated aversion and shame felt by each patient suspected of having the prostate cancer, tumors in an early stage of development are expected to be detected through this way, and a user may easily monitor the prostate cancer anytime and anywhere by checking analysis results in real time.

The above description of the present disclosure is for illustration, and it will be understood that those skilled in the art to which the present disclosure pertains may easily transform the present disclosure in other specific forms without departing from the technical spirit or essential features thereof. Therefore, it should be understood that the above-described exemplary embodiments are illustrative in all respects and not restrictive. For example, each component described as a single type may be implemented in a distributed manner, and similarly, components described as distributed may be implemented in a combined form.

The scope of the present disclosure is indicated by the following claims rather than the above detailed description, and all changes or modifications derived from the meaning and scope of the claims and equivalent concepts should be interpreted as being included in the claims of the present disclosure.

1. A prostate cancer diagnosis system (100) capable of predicting prostate cancer by digitally converting a user's tactile sensation, the system comprising:
a prostate cancer measurement device (110) configured to visualize the tactile sensation as an image by using electrodes applied according to changes in elasticity of prostate tissue to which pressure is applied; and
a mobile device (120) configured to receive visualized prostate tactile sensation numerical data from the prostate cancer measurement device (110), analyze presence or absence of the tissue on the basis of the prostate tactile sensation numerical data, and diagnose the prostate cancer through a result of analyzing the numerical data, so as to output the diagnosis.

2. The system of claim 1, wherein the prostate cancer measurement device (110) is configured to comprise:
a tactile sensor (111) for detecting the tactile sensation according to the changes in the elasticity of the prostate tissue to which the pressure is applied for self-diagnosis of the prostate cancer;
a camera (112) for photographing a region where the tactile sensation is detected by the tactile sensor (111) according to the changes in the elasticity of the prostate tissue to which the pressure is applied, so as to obtain a tactile sensation image;
a Bluetooth module (113) for transmitting the prostate tactile sensation numerical data obtained through the tactile sensor (111) and the camera (112) to the mobile device (120); and
a pressure numerical determination algorithm (114) for determining a pressure numerical value of the tactile sensation according to the changes in the elasticity of the prostate tissue detected by the tactile sensor (111).

3. The system of claim 2, wherein the tactile sensor (111) detects the tactile sensation according to the changes in the elasticity of the prostate tissue to which the pressure is applied for the self-diagnosis of the prostate cancer, and due to contacting a lump during normal probing in which uniform electrical signals are detected, detects a region where the uniform electrical signals are changed into abnormal signals as a prostate nodule region.

4. The system of claim 3, wherein the tactile sensor (111) is configured in an elastic optical waveguide structure in which four white LED units are attached to a glass plate to illuminate an optical waveguide.

5. The system of claim 3, wherein the camera (112) is configured as a near-infrared camera (NIR Camera) that obtains the tactile sensation image by photographing the region where the tactile sensation is detected by the tactile sensor (111) according to the changes in the elasticity of the prostate tissue to which the pressure is applied.

6. The system of claim 3, wherein the Bluetooth module (113) transmits the prostate tactile sensation numerical data obtained through the tactile sensor (111) and camera (112) to the mobile device (120) in real time.

7. The system of claim 1, wherein the mobile device (120) is configured to comprise:
a receiving module (121) for receiving the visualized prostate tactile sensation numerical data from the prostate cancer measurement device (110);
a determination analysis module (122) for analyzing the presence or absence of the tissue on the basis of the prostate tactile sensation numerical data received through the receiving module (121); and
an output module (123) for diagnosing the prostate cancer on the basis of the result of analyzing the numerical data analyzed through the determination analysis module (122), so as to output the diagnosis.

8. The system of claim 7, wherein the mobile device (120) outputs a notification indicating a risk through the output module (123) when the diagnosis of the prostate cancer is determined through the result of analyzing the numerical data analyzed by the determination analysis module (122).

9. A prostate cancer diagnosis method capable of predicting prostate cancer by digitally converting a user's tactile sensation, the method comprising:
(1) visualizing, by a prostate cancer measurement device (110), the tactile sensation as an image by using electrodes applied according to changes in elasticity of prostate tissue to which pressure is applied; and
(2) receiving, by a mobile device (120), visualized prostate tactile sensation numerical data from the prostate cancer measurement device (110), analyzing presence or absence of the tissue on the basis of the prostate tactile sensation numerical data, and diagnosing the prostate cancer through a result of analyzing the numerical data, so as to output the diagnosis.

10. The method of claim 9, wherein the prostate cancer measurement device (110) is configured to comprise:
a tactile sensor (111) for detecting the tactile sensation according to the changes in the elasticity of the prostate tissue to which the pressure is applied for self-diagnosis of the prostate cancer;
a camera (112) for photographing a region where the tactile sensation is detected by the tactile sensor (111) according to the changes in the elasticity of the prostate tissue to which the pressure is applied, so as to obtain a tactile sensation image;
a Bluetooth module (113) for transmitting the prostate tactile sensation numerical data obtained through the tactile sensor (111) and the camera (112) to the mobile device (120); and
a pressure numerical determination algorithm (114) for determining a pressure numerical value of the tactile sensation according to the changes in the elasticity of the prostate tissue detected by the tactile sensor (111).

11. The method of claim 10, wherein the tactile sensor (111) detects the tactile sensation according to the changes in the elasticity of the prostate tissue to which the pressure is applied for the self-diagnosis of the prostate cancer, and due to contacting a lump during normal probing in which uniform electrical signals are detected, detects a region where the uniform electrical signals are changed into abnormal signals as a prostate nodule region.

12. The method of claim 11, wherein the tactile sensor (111) is configured in an elastic optical waveguide structure in which four white LED units are attached to a glass plate to illuminate an optical waveguide.

13. The method of claim 11, wherein the camera (112) is configured as a near-infrared camera (NIR Camera) that obtains the tactile sensation image by photographing the region where the tactile sensation is detected by the tactile sensor (111) according to the changes in the elasticity of the prostate tissue to which the pressure is applied.

14. The method of claim 11, wherein the Bluetooth module (113) transmits the prostate tactile sensation numerical data obtained through the tactile sensor (111) and camera (112) to the mobile device (120) in real time.

15. The method of claim 9, wherein the mobile device (120) is configured to comprise:
a receiving module (121) for receiving the visualized prostate tactile sensation numerical data from the prostate cancer measurement device (110);
a determination analysis module (122) for analyzing the presence or absence of the tissue on the basis of the prostate tactile sensation numerical data received through the receiving module (121); and
an output module (123) for diagnosing the prostate cancer on the basis of the result of analyzing the numerical data analyzed through the determination analysis module (122), so as to output the diagnosis.

16. The method of claim 15, wherein the mobile device (120) outputs a notification indicating a risk through the output module (123) when the diagnosis of the prostate cancer is determined through the result of analyzing the numerical data analyzed by the determination analysis module (122).
